(19) United States
(12) Patent Application Publication
(10) Pub. No.: US 2010/0331703 A1
(43) Pub. Date: Dec. 30, 2010

(54) ULTRASOUND TRANSDUCER DRIVE CIRCUIT AND ULTRASOUND DIAGNOSTIC APPARATUS

(76) Inventor: Shinichi Amemiya, Tokyo (JP)

Correspondence Address: PATRICK W. RASCHE (20459);ARMSTRONG TEASDALE LLP, 7700 Forsyth Boulevard, Suite 1800, St. Louis, MO, 63105, US

(21) Appl. No.: 12/822,996

(22) Filed: Jun. 24, 2010

(30) Foreign Application Priority Data
Jun. 26, 2009 (JP) 2009-151843

(51) Int. Cl. A61B 8/00

(52) U.S. Cl. 600/459

(57) ABSTRACT

An ultrasound transducer drive circuit includes an output line connected to an ultrasound transducer, a plurality of switching circuits each of which turns on and off a connection of a power supply unit with the output line, a plurality of ground clamp circuits each of which turns on and off a connection of the output line with ground, and a control signal output unit which outputs control signals for turning on and off the switching circuits and the ground clamp circuits. The switching circuits are different in impedance from one another, and the ground clamp circuits are different in impedance from one another.

### CROSS REFERENCE TO RELATED APPLICATIONS

This application claims the benefit of Japanese Patent Application No. 2009-151843 filed Jun. 26, 2009, which is hereby incorporated by reference in its entirety.

### BACKGROUND OF THE INVENTION

The present invention relates to an ultrasound transducer drive circuit that supplies a voltage for driving an ultrasound transducer, and an ultrasound diagnostic apparatus equipped with the ultrasound transducer drive circuit.

An ultrasound diagnostic apparatus transmits ultrasound to a subject from an ultrasound probe and receives echoes thereof by the ultrasound probe thereby to generate an ultrasound image in the subject. The ultrasound probe is equipped with an ultrasound transducer composed of a piezoelectric material such as piezoelectric ceramics, and the ultrasound transducer is supplied with each voltage from an ultrasound transducer drive circuit to transmit ultrasound (refer to, for example, Japanese Unexamined Patent Publication No. Hei 11 (1999)-290321).

On the other hand, the ultrasound transducer drive circuit may be a circuit capable of outputting voltages of plural levels. An ultrasound diagnostic apparatus equipped with such an ultrasound transducer drive circuit is capable of generating an arbitrary waveform as a voltage waveform supplied to the ultrasound transducer.

In the ultrasound transducer drive circuit capable of generating the arbitrary waveform as described above, voltages of plural levels have heretofore been outputted by using a class A amplifier. Power consumption, however, increases when the class A amplifier is used. Resistors relatively large in resistance value were needed as resistors for the class A amplifier, thereby making IC implementation difficult and making it impossible to bring it to low cost.

It is desirable that the problem described previously is solved.

### BRIEF DESCRIPTION OF THE INVENTION

The invention according to a first aspect provides an ultrasound transducer drive circuit including an output line connected to an ultrasound transducer, a plurality of switching circuits each of which turns on and off a connection of a power supply unit with the output line, a plurality of ground clamp circuits each of which turns on and off a connection of the output line with ground, and a control signal output unit which outputs control signals for turning on and off the switching circuits and the ground clamp circuits, wherein the switching circuits are different in impedance from one another, and the ground clamp circuits are different in impedance from one another.

The invention according to a second aspect provides an ultrasound transducer drive circuit according to the first aspect, wherein the switching circuits are connected in parallel to one another, wherein the ground clamp circuits are connected in parallel to one another and also connected in parallel to the ultrasound transducer, and wherein the switching circuits and the ground clamp circuits are respectively connected in series with one another.

The invention according to a third aspect provides an ultrasound transducer drive circuit according to the first or second aspect, wherein the impedances of the switching circuits and the ground clamp circuits are set in such a manner that the voltage of the output line has linearity with respect to each of the control signals.

The invention according to a fourth aspect provides an ultrasound transducer drive circuit according to any one of the first through third aspects, wherein the impedances of the switching circuits and the impedances of the ground clamp circuits are respectively set to 1.8ⁿ to 2.2ⁿ (where n: whole number) times the impedance of the switching circuit taken as a reference and the impedance of the ground clamp circuit taken as a reference.

The invention according to a fifth aspect provides an ultrasound transducer drive circuit according to any one of the first through fourth aspects, wherein the impedance of any of the switching circuits and the impedance of any of the ground clam circuits are equal to each other.

The invention according to a sixth aspect provides an ultrasound transducer drive circuit according to any one of the first through fifth aspects, wherein the control signal output unit outputs control signals in such a manner that one of the switching circuits and the ground clamp circuits equal to each other in the order of magnitudes of the impedances of the switching circuits and the order of magnitudes of the impedances of the ground clamp circuits is turned on, and the other thereof is turned off.

The invention according to a seventh aspect provides an ultrasound transducer drive circuit according to any one of the first through sixth aspects, wherein there is provided a correction device for bringing the voltage of the output line to a desired voltage.

The invention according to an eighth aspect provides an ultrasound transducer drive circuit according to the seventh aspect, wherein the correction device includes a lookup table or a correction computer which sets each control signal outputted from the control signal output unit as a control signal capable of obtaining a desired output voltage and outputs the control signal to each of the switching circuits and the ground clamp circuits.

The invention according to a ninth aspect provides an ultrasound transducer drive circuit according to any one of the first through eighth aspects, wherein there are provided transistors each used as a switching element of each of the switching circuits, and wherein the correction device includes sub-transistors connected in parallel with the corresponding transistor as a subarray and smaller in on resistance than the transistor.

The invention according to a tenth aspect provides an ultrasound transducer drive circuit according to any one of the first through ninth aspects, wherein at least one of the ground clamp circuits is always brought to an off state.

The invention according to an eleventh aspect provides an ultrasound transducer drive circuit according to any one of the first through tenth aspects, wherein there are provided a plurality of sets of switching circuit parts comprised of a plurality of the switching circuits different in impedance, and wherein the switching circuit parts are respectively inputted with different power supply voltages each corresponding to an ultrasound transmission mode.

The invention according to a twelfth aspect provides an ultrasound diagnostic apparatus including the ultrasound transducer drive circuit according to the invention of any of the first through eleventh aspects.

According to the invention, the voltage of the output line can be changed by changing on/off states of the switching circuits different in impedance and the ground clamp circuits different in impedance without using a class A amplifier. Therefore, power consumption can be reduced as compared with the use of the class A amplifier, and IC implementation is also easy and can be suppressed to low cost.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing a configuration of an ultrasound diagnostic apparatus according to the invention.

FIG. 2 is a block diagram showing a configuration of an ultrasound transducer drive circuit employed in the ultrasound diagnostic apparatus shown in FIG. 1.

FIG. 3 is a block diagram showing a schematic configuration of a multilevel pulser employed in the ultrasound transducer drive circuit shown in FIG. 2.

FIG. 4 is a circuit diagram showing a schematic configuration of a P-side switching circuit part in the multilevel pulser shown in FIG. 3.

FIG. 5 is a circuit diagram showing a schematic configuration of an N-side switching circuit part in the multilevel pulser shown in FIG. 3.

FIG. 6 is a circuit diagram showing a schematic configuration of a P-side active ground clamp circuit part in the multilevel pulser shown in FIG. 3.

FIG. 7 is a circuit diagram showing a schematic configuration of an N-side active ground clamp circuit part in the multilevel pulser shown in FIG. 3.

FIG. 8 is a diagram for describing operations of the P-side switching circuit part and the P-side active ground clamp circuit part.

FIG. 9 is a diagram for describing control signals inputted to their corresponding P-side switching circuits and P-side active ground clamp circuits.

FIG. 10 is a diagram showing states of the P-side switching circuit part and the P-side active ground clamp circuit part when a control signal “00000” is outputted from a transmitting unit.

FIG. 11 is a diagram showing states of the P-side switching circuit part and the P-side active ground clamp circuit part when a control signal “00001” is outputted from the transmitting unit.

FIG. 12 is a diagram showing a graph of a relationship between a control signal and a positive output voltage.

FIG. 13 is a circuit diagram showing impedance components of the multilevel pulser when the control signal “00001” is outputted from the transmitting unit.

FIG. 14 is a circuit diagram showing impedance components of the multilevel pulser when a control signal “00010” is outputted from the transmitting unit.

FIG. 15 is a diagram showing a graph of a relationship between a control signal and a negative output voltage.

FIG. 16 is a block diagram showing an LUT employed in a first modification of the first embodiment.

FIG. 17 is a diagram showing a relationship between a control signal outputted from the transmitting unit and an output voltage in the first modification of the first embodiment.

FIG. 18 is a circuit diagram showing a first P-side switching circuit employed in a second modification of the first embodiment.

FIG. 19 is a diagram showing a graph of a relationship between a control signal and a positive output voltage employed in a third modification of the first embodiment.

FIG. 20 is a block diagram showing a schematic configuration of a multilevel pulser employed in a second embodiment of the invention.

FIG. 21 is a circuit diagram showing a schematic diagram of a P-side intermediate voltage switching circuit part in the multilevel pulser shown in FIG. 20.

FIG. 22 is a circuit diagram showing a schematic configuration of an N-side intermediate voltage switching circuit part in the multilevel pulser shown in FIG. 20.

FIG. 23 is a block diagram showing a schematic configuration of a multilevel pulser employed in a third embodiment of the invention.

FIG. 24 is a block diagram showing a schematic configuration of a multilevel pulser employed in a fourth embodiment of the invention.

FIG. 25 is a circuit diagram showing a schematic configuration of another example of a P-side active ground clamp circuit part.

FIG. 26 is a circuit diagram showing a schematic configuration of another example of an N-side active ground clamp circuit part.

### DETAILED DESCRIPTION OF THE INVENTION

Preferred embodiments of the invention will hereinafter be described in detail based on the accompanying drawings.

### First Embodiment

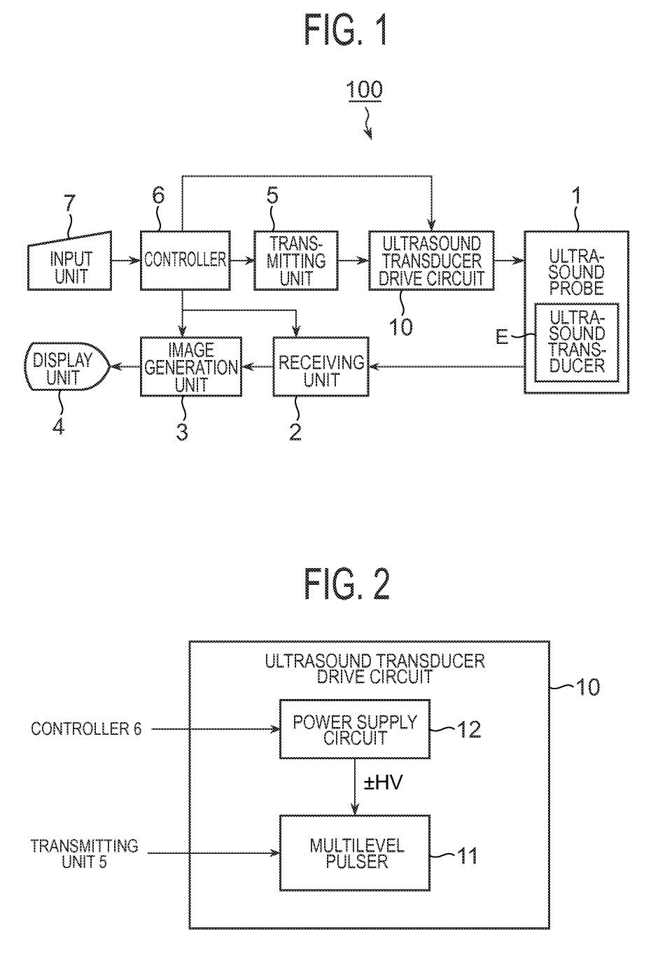

A first embodiment will first be described based on FIGS. 1 through 15. As shown in FIG. 1, an ultrasound diagnostic apparatus 100 has an ultrasound transducer E. The ultrasound diagnostic apparatus 100 has a number of the ultrasound transducers E. The ultrasound diagnostic apparatus 100 has an ultrasound probe 1 which transmits ultrasound to within a subject through the ultrasound transducer E and receives each ultrasound echo from within a subject, a receiving unit 2 which generates and outputs a sound ray signal, based on each ultrasound echo, an image generation unit 3 which generates an ultrasound image, based on the sound ray signal, a display unit 4 which displays the ultrasound image thereon, an ultrasound transducer drive circuit 10 which drives the ultrasound transducer E to transmit ultrasound, a transmitting unit 5 which inputs each signal to be transmitted to the ultrasound transducer drive circuit 10, a controller 6 which controls the entire apparatus, and an input unit 7 for causing an operator to operate.

As shown in FIG. 2, the ultrasound transducer drive circuit 10 is equipped with a multilevel pulser 11 which supplies a drive voltage to the ultrasound transducer E, and a power supply circuit 12 which supplies a power supply voltage ±HV to the multilevel pulser 11. The power supply circuit 12 is one example illustrative of an embodiment of a power supply unit employed in the invention.

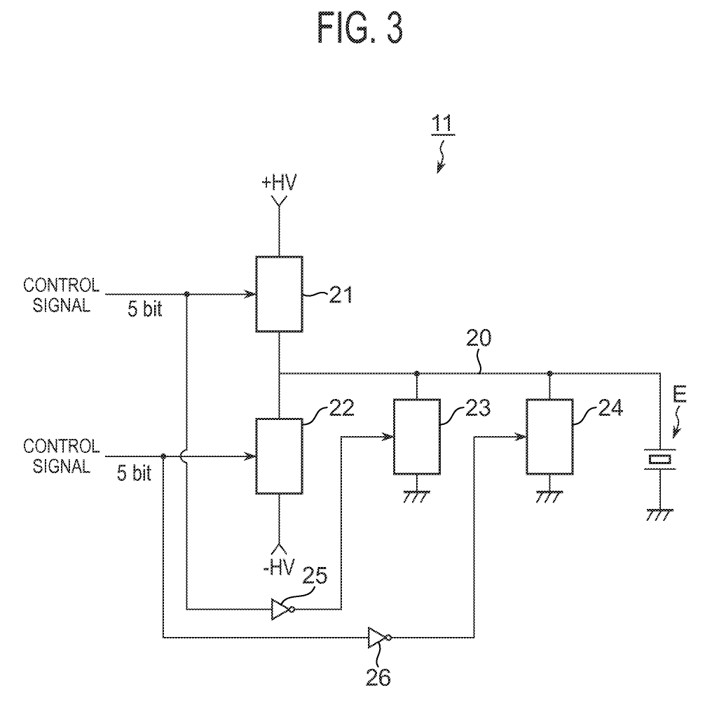

As shown in FIG. 3, the multilevel pulser 11 has an output line 20 connected to the ultrasound transducer E, a P-side switching circuit part 21 connected between the power supply voltage +HV of the power supply unit 12 and the output line 20, an N-side switching circuit part 22 connected between the power supply voltage −HV of the power supply unit 12 and the output line 20, and a P-side active ground clamp circuit part 23 and an N-side active ground clamp circuit part 24 connected between the output line 20 and ground.

The P-side switching circuit part 21, the N-side switching circuit part 22, and the P-side active ground clamp circuit part 23 and N-side active ground clamp circuit part 24 are respectively inputted with 5-bit control signals from the transmitting unit 5. Respective transistors Q1 through Q20 to be described later are turned on and off according to the control signals. Incidentally, the transmitting unit 5 includes a drive circuit for driving the transistors Q1 through Q20 and is one example illustrative of an embodiment of a control signal output unit employed in the invention.

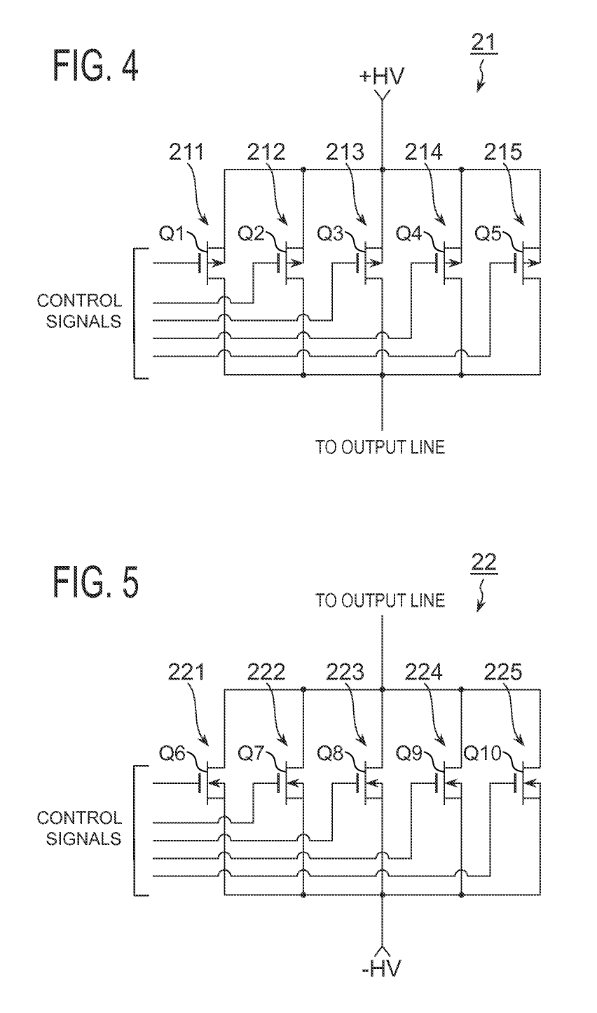

As shown in FIG. 4, the P-side switching circuit part 21 includes a first P-side switching circuit 211, a second P-side switching circuit 212, a third P-side switching circuit 213, a fourth P-side switching circuit 214 and a fifth P-side switching circuit 215. These first through fifth P-side switching circuits 211 through 215 are connected in parallel to one another between the power supply voltage +HV and the output line 20.

The P-side switching circuits 211 through 215 respectively have the transistors Q1, Q2, Q3, Q4 and Q5. The transistors Q1 through Q5 are respectively FETs (Field Effect Transistors) and turned on and off according to their corresponding control signals from the transmitting unit 5. The P-side switching circuits 211 through 215 respectively turn on and off the connection of the power supply voltage +HV and the output line 20 by means of the transistors Q1 through Q5 and are one example illustrative of embodiments of switching circuits employed in the invention.

The transistors Q1 through Q5 are different from one another in on resistance. Thus, the P-side switching circuits 211 through 215 are different in impedance from one another. Described specifically, when the on resistance of the transistor Q1 is assumed to be X (Ω), the on resistance of the transistor Q2 becomes 2X (Ω), the on resistance of the transistor Q3 becomes 4X (Ω), the on resistance of the transistor Q4 becomes 8X (Ω), and the on resistance of the transistor Q5 becomes 16X (Ω). That is, the on resistances of the transistors Q2 through Q5 become resistance values of 2ⁿ (where n: whole number) times the on resistance (on resistance taken as a reference) of the transistor Q1.

As shown in FIG. 5, the N-side switching circuit 22 includes a first N-side switching circuit 221, a second N-side switching circuit 222, a third N-side switching circuit 223, a fourth N-side switching circuit 224 and a fifth N-side switching circuit 225. These first through fifth N-side switching circuits 221 through 225 are connected in parallel to one another between the power supply voltage −HV and the output line 20.

The N-side switching circuits 221 through 225 respectively have a transistor Q6, a second transistor Q7, a third transistor Q8, a fourth transistor Q9 and a fifth transistor Q10. The transistors Q6 through Q10 are FETs and are turned on and off according to their corresponding control signals from the transmitting unit 5. The N-side switching circuits 221 through 225 respectively turn on and off the connection of the power supply voltage −HV and the output line 20 by means of the transistors Q6 through Q10 and are one example illustrative of embodiments of switching circuits employed in the invention.

The transistors Q6 through Q10 are different from one another in on resistance. Thus, the N-side switching circuits 221 through 225 are different in impedance from one another. Described specifically, the on resistance of the transistor Q6 is X (Ω) identical to the on resistance of the transistor Q1, the on resistance of the transistor Q7 is 2X (Ω) identical to the on resistance of the transistor Q2, and the on resistance of the transistor Q8 is 4X (Ω) identical to the on resistance of the transistor Q3. The on resistance of the transistor Q9 becomes 8X (Ω) identical to the on resistance of the transistor Q4, and the on resistance of the transistor Q10 becomes 16X (Ω) identical to the on resistance of the transistor Q5. Namely, the on resistances of the transistors Q7 through Q10 also become resistance values of 2′ (where n: whole number) times the on resistance (on resistance taken as a reference) of the transistor Q6.

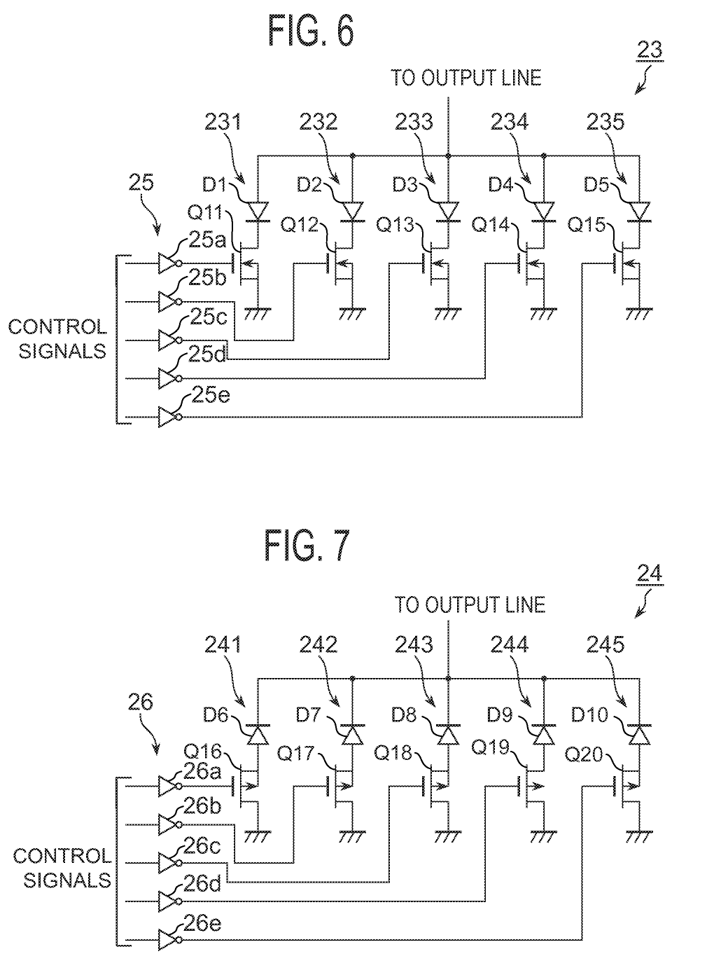

As shown in FIG. 6, the P-side active ground clamp circuit part 23 includes a first P-side active ground clamp circuit 231, a second P-side active ground clamp circuit 232, a third P-side active ground clamp circuit 233, a fourth P-side active ground clamp circuit 234 and a fifth P-side active ground clamp circuit 235. These first through fifth P-side active ground clamp circuits 231 through 235 are connected in parallel to one another between the output line 20 and ground.

The P-side active ground clamp circuits 231 through 235 have transistors Q11, Q12, Q13, Q14 and Q15 and include diodes D1, D2, D3, D4 and D5, respectively. These transistors Q11 through Q15 and diodes D1 through D5 are series-connected to one another.

The transistors Q11 through Q15 are FETs and are turned on and off according to their corresponding control signals from the transmitting unit 5. The P-side active ground clamp circuits 231 through 235 respectively turn on and off the connection of the output line 20 with ground by means of the transistors Q11 through Q15 and are one example illustrative of embodiments of ground clamp circuits employed in the invention.

The control signals inputted from the transmitting unit 5 to the P-side switching circuit part 21 are inverted by inverters 25a, 25b, 25c, 25d and 25e (shown collectively as inverter 25 in FIGS. 3 and 6), followed by being inputted to the transistors Q11 through Q15 respectively.

The transistors Q11 through Q15 are different in on resistance from one another. Thus, the P-side active ground clamp circuits 231 through 235 are different in impedance from one another. Described specifically, the on resistance of the transistor Q11 is Y (Ω), the on resistance of the transistor Q12 is 2Y (Ω), and the on resistance of the transistor Q13 is 4Y (Ω). The on resistance of the transistor Q14 becomes 8Y (Ω), and the on resistance of the transistor Q15 becomes 16Y (Ω). Namely, the on resistances of the transistors Q12 through Q15 become resistance values of 2ⁿ (where n: whole number) times the on resistance (on resistance taken as a reference) of the transistor Q11.

As shown in FIG. 7, the N-side active ground clamp circuit part 24 includes a first N-side active ground clamp circuit 241, a second N-side active ground clamp circuit 242, a third N-side active ground clamp circuit 243, a fourth N-side active ground clamp circuit 244 and a fifth N-side active ground clamp circuit 245. These first through fifth N-side active ground clamp circuits 241 through 245 are connected in parallel to one another between the output line 20 and ground.

The N-side active ground clamp circuits 241 through 245 have transistors Q16, Q17, Q18, Q19 and Q20 and include diodes D6, D7, D8, D9 and D10, respectively. These transistors Q16 through Q20 and diodes D6 through D10 are series-connected to one another.

The transistors Q16 through Q20 are FETs and are turned on and off according to their corresponding control signals from the transmitting unit 5. The N-side active ground clamp circuits 241 through 245 respectively turn on and off the connection of the output line 20 with ground by means of the transistors Q16 through Q20 and are one example illustrative of embodiments of ground clamp circuits employed in the invention.

The control signals inputted from the transmitting unit 5 to the N-side switching circuit part 22 are inverted by inverters 26a, 26b, 26c, 26d and 26e (shown collectively as inverter 26 in FIGS. 3 and 7), followed by being inputted to the transistors Q16 through Q20 respectively.

The transistors Q16 through Q20 are different from one another in on resistance. Thus, the N-side active ground clamp circuits 241 through 245 are different in impedance from one another. Described specifically, the on resistance of the transistor Q16 is Y (Ω) identical to the on resistance of the transistor Q11, the on resistance of the transistor Q17 is 2Y (Ω) identical to the on resistance of the transistor Q12, and the on resistance of the transistor Q18 is 4Y (Ω) identical to the on resistance of the transistor Q13. The on resistance of the transistor Q19 becomes 8Y (Ω) identical to the on resistance of the transistor Q14, and the on resistance of the transistor Q20 becomes 16Y (Ω) identical to the on resistance of the transistor Q15. Namely, the on resistances of the transistors Q17 through Q20 also become resistance values of 2ⁿ (where n: whole number) times the on resistance (on resistance taken as a reference) of the transistor Q16.

Here, X=Y may be taken or X≠Y may be taken. When, however, X=Y, the voltage of the output line 20 can be set to one having linearity as will be described later.

The operation of the ultrasound transducer drive circuit 10 according to the present embodiment will now be explained. In the ultrasound transducer drive circuit 10, the P-side switching circuit part 21 and the P-side active ground clamp circuit 23 connected in series with each other are operated when a positive voltage is supplied to the ultrasound transducer E. On the other hand, when a negative voltage is supplied to the ultrasound transducer E, the N-side switching circuit part 22 and the N-side active ground clamp circuit part 24 connected in series with each other are operated. Thus, when the P-side switching circuit part 21 and the P-side active ground clamp circuit part 23 are operated, the N-side switching circuit part 22 and the N-side active ground clamp circuit part 24 are not operated so that the transistors Q6 through Q10 and the transistors Q16 through Q20 are respectively brought to an off state. On the other hand, when the N-side switching circuit part 22 and the N-side active ground clamp circuit part 24 are operated, the P-side switching circuit part 21 and the P-side active ground clamp circuit part 23 are not operated so that the transistors Q1 through Q5 and the transistors Q11 through Q15 are respectively brought to an off state.

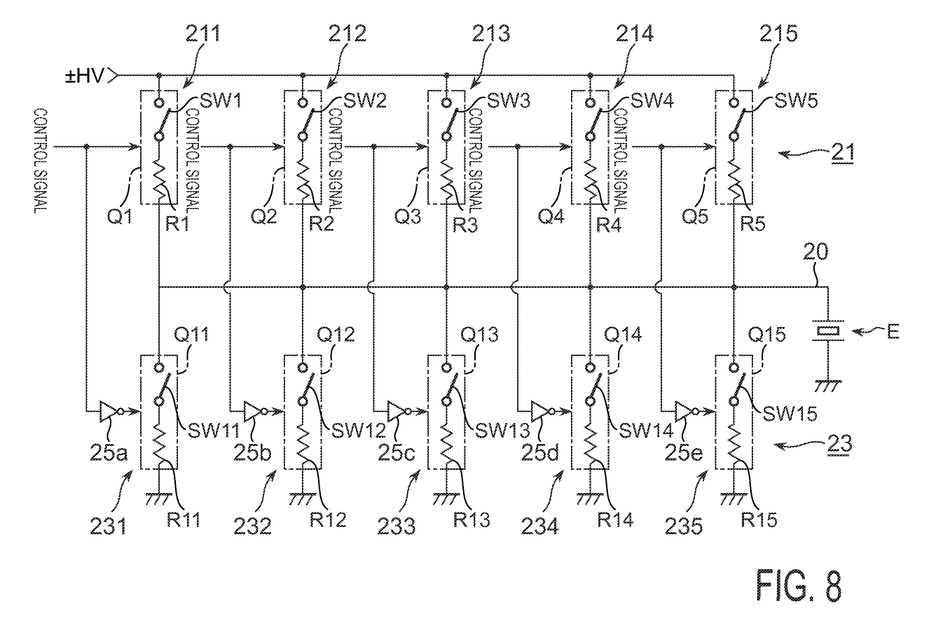

The operations of the P-side switching circuit part 21 and the P-side active ground clamp circuit part 23 will be described based on FIGS. 8 through 11. In the P-side switching circuit part 21 shown in FIG. 8, the transistors Q1 through Q5 are represented by switches SW1, SW2, SW3, SW4 and SW5 and resistors R1, R2, R3, R4 and R5 respectively. Similarly, in the P-side active ground clamp circuit part 23, the transistors Q11 through Q15 are represented by switches SW11, SW12, SW13, SW14 and SW15 and resistors R11, R12, R13, R14 and R15 respectively.

The resistors R1 through R5 indicate the on resistances of the transistors Q1 through Q5 respectively. Namely, R1=X, R2=2X, R3=4X, R4=8X and R5=16X . Further, the resistors R11 through R15 indicate the on resistances of the transistors Q11 through Q15 respectively. Namely, R11=Y, R12=2Y, R13=4Y, R14=8Y and R15=16Y. X and Y are, however, assumed to be X=Y here. Thus, R1=R11, R2=R12, R3=R13, R4=R14 and R5=R15.

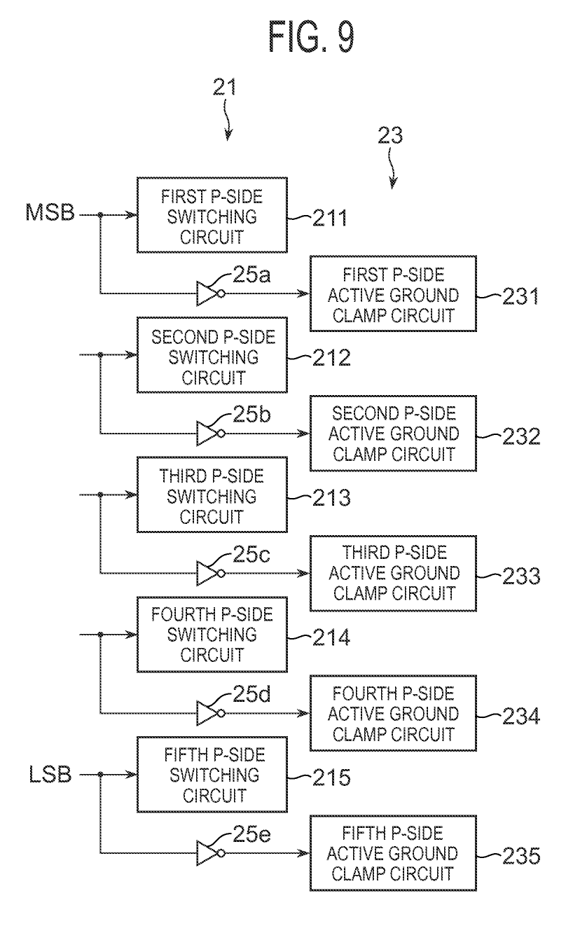

The P-side switching circuit part 21 and the P-side active ground clamp circuit part 23 are respectively inputted with 5-bit control signals from the transmitting unit 5, so that the transistors Q1 through Q5 and the transistors Q11 through Q15 are turned on and off (the switches SW1 through SW5 and the switches SW11 through SW15 are turned on and off). As shown in FIG. 9, the P-side switching circuits 211 through 215 are respectively inputted with such control signals that the first P-side switching circuit 211 is brought to the most significant bit (MSB) and the fifth P-side switching circuit 215 is brought to the least significant bit (LSB). Further, the P-side active ground clamp circuits 231 through 235 are respectively inputted with such control signals that the first P-side active ground clamp circuit 231 is brought to the most significant bit and the fifth P-side active ground clamp circuit 235 is brought to the least significant bit. However, the control signals inputted to the P-side switching circuits 211 through 215 are inverted by their corresponding inverters 25a, 25b, 25c, 25d and 25e, followed by being inputted to the P-side active ground clamp circuits 231 through 235.

When the P-side switching circuits 211 through 215 and the P-side active ground clamp circuits 231 through 235 are respectively inputted with signals each having an L level corresponding to a level low in voltage as control signals, the transistors Q1 through Q5 and the transistors Q11 through Q15 are turned off On the other hand, when they are respectively inputted with signals each having an H level corresponding to a level high in voltage as control signals, the transistors Q1 through Q5 and the transistors Q11 through Q15 are turned on.

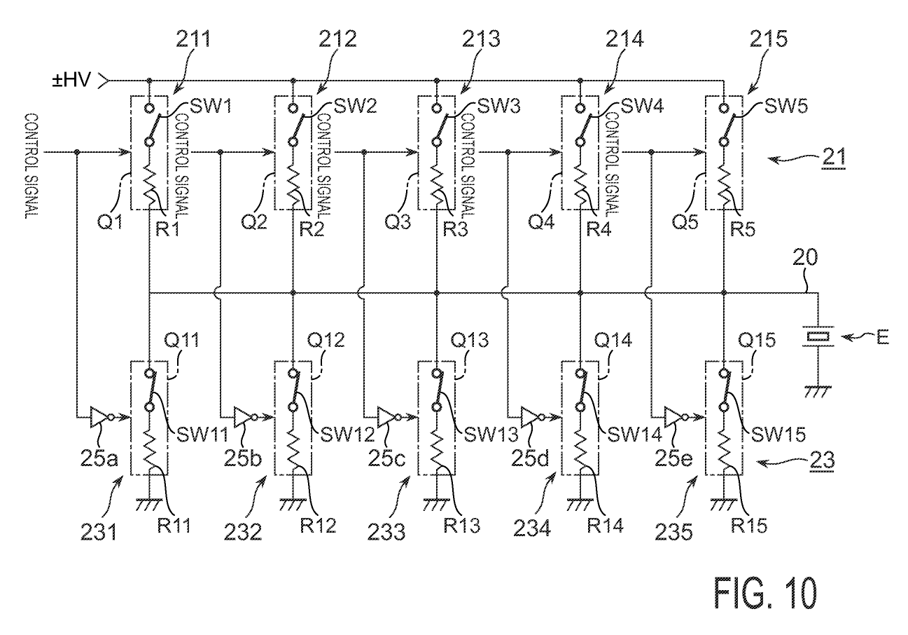

Assuming that the signals each having the L level are represented by “0” and the signals each having the H level are represented by “1”, 32 types of controls signals corresponding to “00000” through “11111” are inputted to the P-side switching circuit part 21 and the P-side active ground clamp circuit part 23 as 5-bit control signals. When, for example, the control signal “00000” is outputted from the transmitting unit 5, the control signal of “00000” is inputted to the P-side switching circuit part 21 and its inverted control signal “11111” is inputted to the P-side active ground clamp circuit part 23, such a state as shown in FIG. 10 is reached. Namely, the transistors Q1 through Q5 are all turned off, whereas the transistors Q11 through Q15 are all turned on.

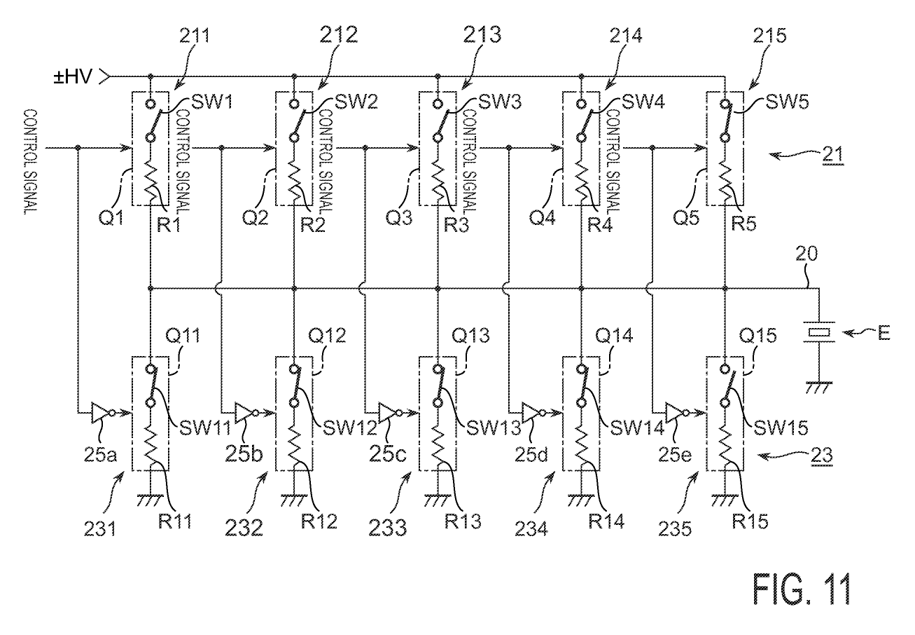

When the control signal “00001” is outputted from the transmitting unit 5, the control signal of “00001” is inputted to the P-side switching circuit part 21, and its inverted control signal “11110” is inputted to the P-side active ground clamp circuit part 23, such a state as shown in FIG. 11 is reached. Namely, the transistors Q1 through Q4 and the transistor Q15 are turned off, whereas the transistor Q5 and the transistors Q11 through Q14 are all turned on.

Changing the states of on/off of the transistors Q1 through Q5 in this way makes it possible to change the synthetic impedance of the P-side switching circuit part 21. Also changing the states of on/off of the transistors Q11 through Q15 makes it possible to change the synthetic impedance of the P-side active ground clamp circuit part 23. The voltage to be supplied to the ultrasound transducer E, i.e., the voltage (output voltage) of the output line 20 can be changed by changing the synthetic impedance of the P-side switching circuit part 21 and that of the P-side active ground clamp circuit part 23 in this way.

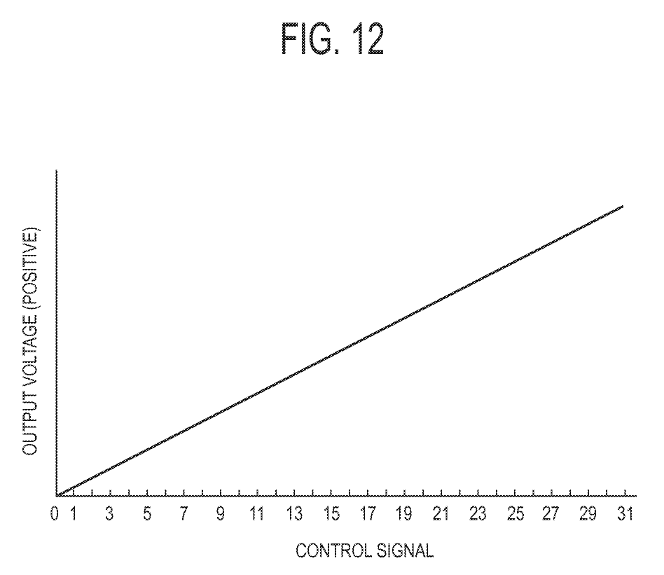

The relationship between the control signals inputted to the P-side switching circuit part 21 and the P-side active ground clamp circuit part 23 and the output voltage will now be explained based on FIG. 12. In a graph shown in FIG. 12, the horizontal axis indicates 5-bit control signals expressed in binary numbers in the form of decimal numbers (for example, the control signal “00001” is expressed in 1, and the control signal “11111” is expressed in 31). As shown in FIG. 12, the relationship between each input control signal and the voltage (output voltage) supplied to the ultrasound transducer E becomes linear. It is necessary, however, to set the on resistances of the transistors Q1 through Q5 to X, 2X, 4X, 8X and 16X respectively, set the on resistances of the transistors Q11 through Q15 to Y, 2Y, 4Y, 8Y and 16Y respectively, and further set the condition of X=Y as described above.

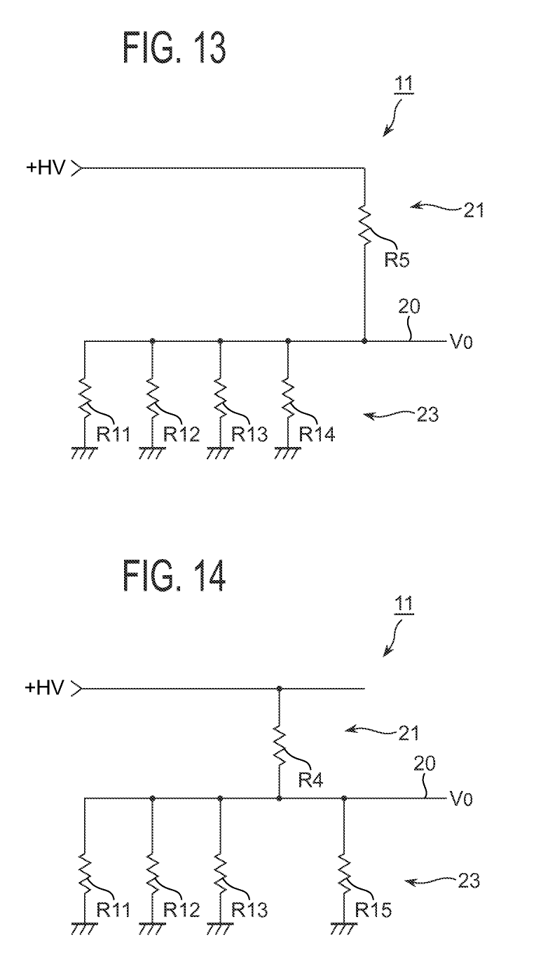

A detailed description will be made of the point in which the relationship between the control signal and the output voltage becomes linear. When, for example, the control signal “00001” is outputted from the transmitting unit 5, the control signal “00001” is inputted to the P-side switching circuit part 21 and its inverted signal “11110” is inputted to the P-side active ground clamp circuit part 23, the multilevel pulser 11 becomes a circuit having such impedance components as shown in FIG. 13 (the diodes D1 through D5 are considered in disregard therefore). Namely, R11, R12, R13 and R14 connected in parallel to one another are connected in series with R5. Now considering where the output voltage is assumed to be Vo, the power supply voltage is assumed to be HV, the synthetic impedance of the P-side switching circuit part 21 is assumed to be RS, the synthetic impedance of the P-side active ground clamp circuit part 23 is assumed to be RG, and the resistive component of the ultrasound transducer E is ignored, the following equation is established:

Vo={RG/(RS+RG)}·HV (1)

Thus, when RS and RG are determined in the circuit shown in FIG. 13, RS is first brought to RS =16. Further, RG is brought to RG =(8/15) Y. Substituting RS =16 and RG =8/15 into the equation (1) in disregard for X and Y because of X=Y yields Vo =(1/31) HV.

When, for example, the control signal “00010” is outputted from the transmitting unit 5, the control signal “00010” is inputted to the P-side switching circuit part 21, and “11101” indicative of its inverted signal is inputted to the P-side active ground clamp circuit part 23, the multilevel pulser 11 is brought to a circuit having such impedance components as shown in FIG. 14 (the diodes D1 through D5 are considered in disregard therefore). Namely, R11, R12, R13 and R15 connected in parallel to one another are connected in series with R4. Thus, RS =8×, and RG =(16/29) Y. Substituting RS =8 and RG =16/29 into the equation (1) in disregard for X and Y because of X=Y yields Vo =(2/31) HV.

Thus, the term of {RG/(RS +RG)} in the equation (1) becomes n/31 (however, except for n=31). n indicates numerals taken when the control signals are represented in decimal numbers. n=1 if, for example, “00001” is taken, n=2 if “00010” is taken, and n=3 if “00011” is taken. Thus, the output voltage Vo becomes larger as the control signal makes a carry, and hence such a linear graph as shown in FIG. 12 is obtained.

The operations of the N-side switching circuit part 22 and the N-side active ground clamp circuit part 24 will next be explained. With the N-side switching circuit part 22 and the N-side active ground clamp circuit part 24 being also inputted with 5-bit control signals, the transistors Q6 through Q10 and the transistors Q16 through Q20 are turned on and off. Consequently, the synthetic impedance of each of the N-side switching circuit part 22 and the N-side active ground clamp circuit part 24 changes, thereby making it possible to change the output voltage.

Incidentally, in the N-side switching circuit part 22, the first N-side switching circuit 221 is brought to the most significant bit, and the fifth N-side switching circuit 225 is brought to the least significant bit. In the N-side active ground clamp circuit part 24 as well, the first N-side active ground clamp circuit 241 is brought to the most significant bit, and the fifth N-side active ground clamp circuit 245 is brought to the least significant bit.

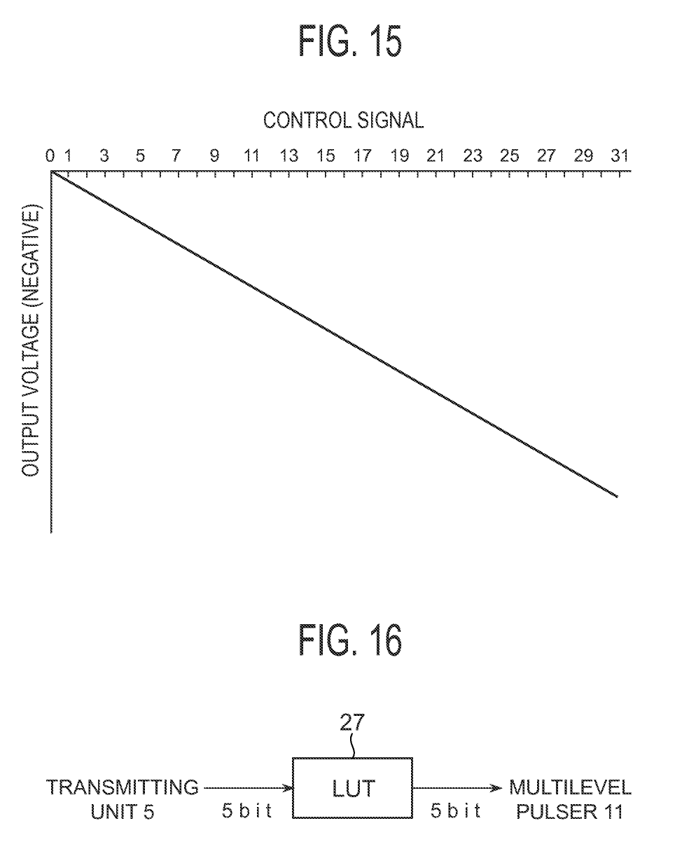

The relationship between the control signals inputted to the N-side switching circuit part 22 and the N-side active ground clamp circuit part 24 and the output voltage will be explained based on FIG. 15. When the N-side switching circuit part 22 and the N-side active ground clamp circuit part 24 are operated, an output voltage equal in absolute value to the output voltage and opposite in sign thereto where the P-side switching circuit part 21 and the P-side active ground clamp circuit part 23 are operated, is obtained. Thus, the relationship between each input control signal and the output voltage becomes linear as shown in FIG. 15.

According to the multilevel pulser 11 of the present embodiment which can obtain the above-described output voltage, a voltage having a desired waveform can be supplied to the ultrasound transducer E by inputting a predetermined control signal. When, for example, a sinusoidal voltage is supplied to the ultrasound transducer E, the P-side switching circuit part 21 and the P-side active ground clamp circuit part 23 are first operated. At this time, “00000”, “00001”, . . . , “11110”, and “11111” are outputted in order as the control signals from the transmitting unit 5 and thereafter “11110”, “11101”, . . . , and “00000” are outputted, thereby operating the P-side switching circuit part 21 and the P-side active ground clamp circuit part 23. Next, the N-side switching circuit part 22 and the N-side active ground clamp circuit part 24 are operated. In a manner similar to the above even at this time, “00000”, “00001”, . . . “11110”, and “11111” are outputted in order as the control signals from the transmitting unit 5 and thereafter “11110”, “11101”, . . . , and “00000” are outputted.

According to the ultrasound transducer drive circuit 10 of the present embodiment described above, since no class A amplifier is used, the power consumption can be reduced than conventional, and IC implementation is also easy and can be suppressed to low cost.

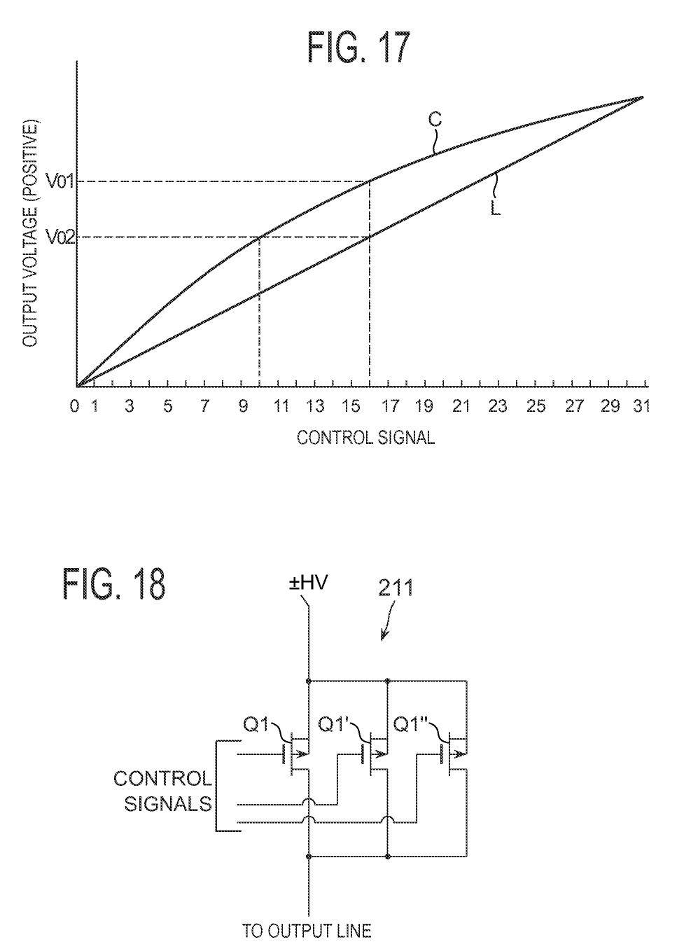

Modifications of the first embodiment will next be described. A first modification will first be described based on FIGS. 16 and 17. In the first modification, control signals outputted from the transmitting unit 5 are table-converted at an LUT (Look Up Table) 27, followed by being outputted to the multilevel pulser 11. The LUT 27 is one example illustrative of an embodiment of a correction device employed in the invention.

At the LUT 27, table conversion is performed in such a manner that a desired output voltage is obtained. Even though, for example, the values of the on resistances of the transistors Q1 through Q20 are of such values that the relationship between each control signal inputted to the multilevel pulser 11 and the output voltage is prevented from becoming linear, the table conversion is performed by the LUT 27 in such a manner that a linear output voltage is obtained. Described concretely, assume that, for example, X≠Y at the transistors Q1 through Q20. Assume in this case that such an output voltage as indicated by a curve C in FIG. 17 is obtained where no table conversion is done at the LUT 27 when the P-side switching circuit part 21 and the P-side active ground clamp circuit part 23 are operated. When the control signal “10000” (16 in the form of the decimal number) is outputted from the transmitting unit 5, the output voltage is brought to Vo1 at the curve C. When it is desired, however, to obtain a linear voltage as the output voltage (straight line L), the output voltage to be obtained when the control signal “10000” is outputted, is Vo2. The output voltage is brought to Vo2 at the curve C when the control signal outputted from the transmitting unit 5 is of “01010” (10 in the form of the decimal number). Thus, when the control signal outputted from the transmitting unit 5 is of “10000”, table conversion is performed thereon at the LUT 27 to output the corresponding control signal “01010” from the LUT 27.

Incidentally, in the first modification, a correction computer to which such a function that a control signal that assumes a desired output voltage is calculated based on the control signal outputted from the transmitting unit 5 is set, may be used instead of the LUT 27.

A second modification will next be explained. The first P-side switching circuit 211 employed in the second modification will be described based on FIG. 18. In the second modification, sub-transistors Q1′ and Q1″ smaller in on resistance than the transistor Q1 are connected in parallel with the transistor Q1 as a subarray in the first P-side switching circuit 211. The on resistances of the sub-transistors Q1′ and Q1″ become about one-tenth the on resistance of the transistor Q1 (subarray structure). The sub-transistors Q1′ and Q1″ are one example illustrative of an embodiment of a correction device employed in the invention.

In the first P-side switching circuit 211 of the second modification, the sub-transistor Q1′ is operated simultaneously with the transistor Q1 with operation similar to the transistor Q1 in an initial state. Namely, when the transistor Q1 is turned on, the sub-transistor Q1′ is also turned on simultaneously. When the transistor Q1 is turned off, the sub-transistor Q1′ is also turned off simultaneously.

When the output voltage obtained when the transistor Q1 and the sub-transistor Q1′ are operated simultaneously as described above, is not brought to a desired voltage, the sub-transistor Q1″ is caused to perform a similar operation simultaneously with the transistor Q1 and the sub-transistor Q1′ or the sub-transistor Q1′ is prevented from operating, in such a manner that the desired voltage is reached. Consequently, the synthetic impedance of the first P-side switching circuit part 21 can be adjusted. Thus, even if the on resistance of the transistor Q1 varies in manufacture, the first P-side switching circuit 211 is brought to a desired synthetic impedance, thereby making it possible to obtain a desired output voltage.

Although the first P-side switching circuit 211 has been explained as above, the P-side switching circuits 212 through 215 other than it also assume a subarray structure similar to the first P-side switching circuit. The N-side switching circuits 221 through 225 also assume a subarray structure similar to the first P-side switching circuit 211.

Instead of the P-side switching circuits 211 through 215, the P-side active ground clamp circuits 231 through 235 may be configured by the subarray structure. Further, as an alternative to the N-side switching circuits 221 through 225, the N-side active ground clamp circuits 241 through 245 may be configured by the subarray structure. Configuring the active ground clamp circuits 231 through 235 and the N-side active ground clamp circuits 241 through 245 by the subarray structure, respectively, in this way makes it possible to bring the impedances of the active ground clamp circuits 231 through 235 and the N-side active ground clamp circuits 241 through 245 to desired impedances and thereby obtain desired output voltages respectively.

A third modification will next be described. In the third modification, the first P-side active ground clamp circuit 231 and the first N-side active ground clamp circuit 241 are respectively always held in an off state. Consequently, power consumption of the first P-side active ground clamp circuit 231 and the first N-side active ground clamp circuit 241 are eliminated, thus making it possible to achieve low power consumption.

For example, an output voltage Vo where the P-side switching circuit part 21 and the P-side active ground clamp circuit part 23 are operated is determined in the third modification. When the control signal outputted from the transmitting unit 5 is of “00001”, for example, the synthetic impedance RS of the P-side switching circuit part 21 and the synthetic impedance RG of the P-side active ground clamp circuit part 23 are RS =16× and RG =(7/8) Y respectively. Accordingly, Vo =(1/15) HV is obtained when these are substituted into the equation (1).

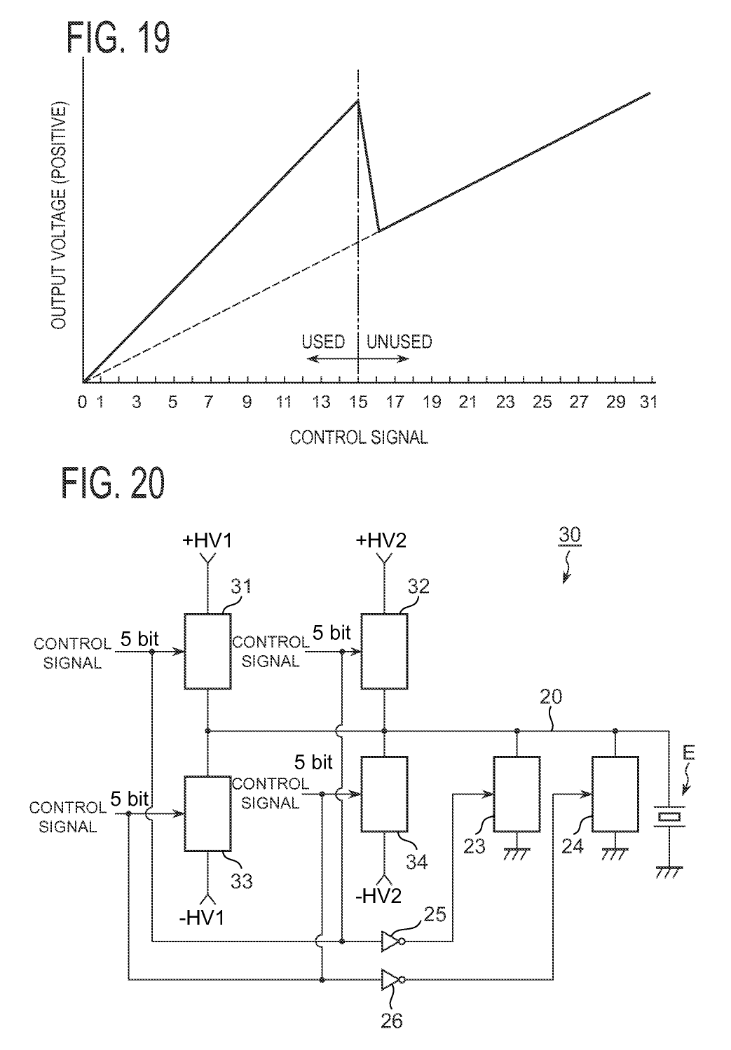

Since RS =8× and RG =(16/13) Y where the control signal outputted from the transmitting unit 5 is of “00010”, Vo =(2/15) HV is obtained when these are substituted into the equation (1). As described above, the output voltage becomes larger between the control signals “00000” and “01111” as compared with each of the above embodiments as shown in FIG. 19. Incidentally, a broken line indicates the output voltage obtained in the above embodiment in FIG. 19.

Since RS =X and RG =(16/15) Y where the control signal outputted from the transmitting unit 5 is of “10000”, Vo =(16/31) HV is obtained when these are substituted into the equation (1), so that the output voltage becomes lower than that at the control signal “01111”. The output voltage becomes equal to that obtained in the above embodiment up to the control signal “11111” after the control signal “10000”.

Since the above-described output voltages are obtained in the third modification, the P-side switching circuit part 21 and the P-side active ground clamp circuit part 23, and the N-side switching circuit part 22 and the N-side active ground clamp circuit part 24 are operated using the control signals “00000” through “01111” without using the control signals “10000” through “11111”.

### Second Embodiment

A second embodiment will next be explained based on FIGS. 20 through 22. The same components as those employed in the first embodiment are, however, respectively given the same reference numerals, and their explanations will therefore be omitted.

A multilevel pulser 30 of the ultrasound transducer drive circuit 10 according to the second embodiment is connected to either the power supply voltage ±HV1 of the power supply unit 12 or the power supply voltage ±HV2 thereof according to a transmission mode such as a B mode or a Doppler mode. Incidentally, ±HV1 indicates the maximum voltage and is used as a power supply voltage for the B mode, for example. On the other hand, ±HV2 indicates an intermediate voltage between ±HV1 and a zero voltage and is used as a power supply voltage for the Doppler mode, for example.

Described specifically, in the multilevel pulser 30 of the present embodiment, as shown in FIG. 20, a P-side maximum voltage switching circuit part 31 is connected between the power supply voltage +HV1 and the output line 20, and a P-side intermediate voltage switching circuit part 32 is connected between the power supply voltage +HV2 and the output line 20. Namely, the multilevel pulser 30 of the present embodiment is provided with positive voltage-side switching circuit parts in pairs. The P-side active ground clamp circuit part 23 is connected between the output line 20 and ground.

Although not illustrated in particular, the P-side maximum voltage switching circuit part 31 assumes the same configuration as the P-side switching circuit part 21 employed in the first embodiment and includes a plurality of switching circuits. The P-side maximum voltage switching circuit part 31 is also inputted with 5-bit control signals outputted from the transmitting unit 5. The 5-bit control signals inputted to the P-side maximum voltage switching circuit part 31 are inverted by the corresponding inverter 25, followed by being inputted to the P-side active ground clamp circuit part 23.

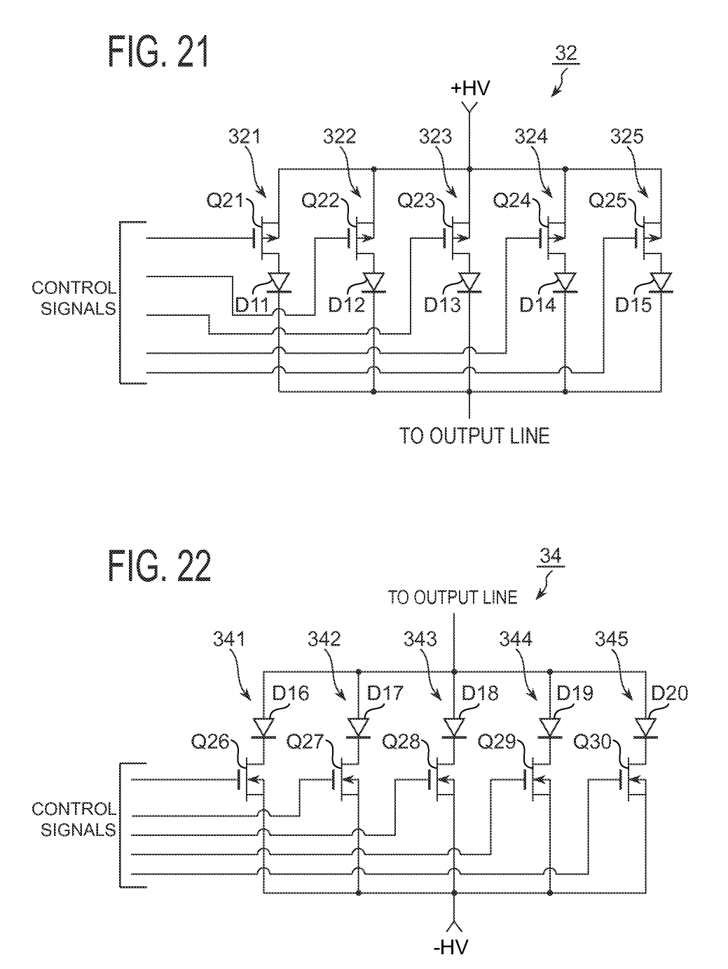

On the other hand, the P-side intermediate voltage switching circuit part 32 has five switching circuits similar to those of the P-side switching circuit part 21, i.e., a first P-side intermediate voltage switching circuit 321, a second P-side intermediate voltage switching circuit 322, a third P-side intermediate voltage switching circuit 323, a fourth P-side intermediate voltage switching circuit 324, and a fifth P-side intermediate voltage switching circuit 325 as shown in FIG. 21. The P-side intermediate voltage switching circuits 321 through 325 are one example illustrative of embodiments of switching circuits employed in the invention.

The P-side intermediate voltage switching circuits 321 through 325 respectively have transistors Q21, Q22, Q23, Q24 and Q25 and diodes D11, D12, D13, D14 and D15. In a manner similar to the first embodiment, the on resistance of the transistor Q21 becomes X (Ω), the on resistance of the transistor Q22 becomes 2X (Ω), the on resistance of the transistor Q23 becomes 4X (Ω), the on resistance of the transistor Q24 becomes 8X (Ω), and the on resistance of the transistor Q25 becomes 16X (Ω).

The transistors Q21 through Q25 are connected to their corresponding anodes of the diodes D11 through D15, and the output line 20 is connected to its corresponding cathodes thereof.

When the P-side maximum voltage switching circuit part 31 and the P-side active ground clamp circuit part 23 are operated here, the voltage (output voltage) of the output line 20 may become higher than the power supply voltage +HV2. For such a case, the diodes D11 through D15 are provided to protect the transistors Q21 through Q25.

The transistors Q21 through Q25 of the P-side intermediate voltage switching circuits 321 through 325 are also inputted with their corresponding control signals outputted from the transmitting unit 5. The 5-bit control signals inputted to the P-side intermediate voltage switching circuit parts 321 through 325 are inverted by the corresponding inverter 25, followed by being inputted to the P-side active ground clamp circuit part 23.

An N-side maximum voltage switching circuit part 33 is connected between the power supply voltage −HV1 and the output line 20, and an N-side intermediate voltage switching circuit part 34 is connected between the power supply voltage −HV2 and the output line 20. Namely, the multilevel pulser 30 of the present embodiment is equipped with negative voltage-side switching circuit parts in pairs. The P-side active ground clamp circuit part 23 is connected between the output line 20 and ground.

Although not illustrated in particular, the N-side maximum voltage switching circuit part 33 assumes a configuration identical to that of the N-side switching circuit part 23 employed in the first embodiment. The N-side maximum voltage switching circuit 33 is also inputted with 5-bit control signals outputted from the transmitting unit 5. The 5-bit control signals inputted to the N-side maximum voltage switching circuit part 33 are inverted by the corresponding inverter 26, followed by being inputted to the N-side active ground clamp circuit part 24.

On the other hand, the N-side intermediate voltage switching circuit part 34 has five switching circuits similar to those of the N-side switching circuit part 22, i.e., a first N-side intermediate voltage switching circuit 341, a second N-side intermediate voltage switching circuit 342, a third N-side intermediate voltage switching circuit 343, a fourth N-side intermediate voltage switching circuit 344, and a fifth N-side intermediate voltage switching circuit 345 as shown in FIG. 22. The N-side intermediate voltage switching circuits 341 through 345 are one example illustrative of embodiments of switching circuits employed in the invention.

The N-side intermediate voltage switching circuits 341 through 345 respectively have transistors Q26, Q27, Q28, Q29 and Q30 and diodes D16, D17, D18, D19 and D₂O. In a manner similar to the first embodiment, the on resistance of the transistor Q26 becomes X (Ω), the on resistance of the transistor Q27 becomes 2X (Ω), the on resistance of the transistor Q28 becomes 4X (Ω), the on resistance of the transistor Q29 becomes 8X (Ω), and the on resistance of the transistor Q30 becomes 16X (Ω).

The output line 20 is connected to its corresponding anodes of the diodes D16 through D20, and the transistors Q26 through Q30 are connected to their corresponding cathodes thereof. Incidentally, the diodes D16 through D20 are also provided to protect the transistors Q26 through Q30.

The N-side intermediate voltage switching circuits 341 through 345 are also inputted with their corresponding control signals outputted from the transmitting unit 5. The 5-bit control signals inputted to the N-side intermediate voltage switching circuit parts 341 through 345 are inverted by the corresponding inverter 26, followed by being inputted to the N-side active ground clamp circuit part 24.

When the positive voltage is supplied to the ultrasound transducer E where the power supply voltage ±HV1 is used in the multilevel pulser 30 of the present embodiment, the corresponding control signals are outputted from the transmitting unit 5 in a manner similar to the first embodiment to operate the P-side maximum voltage switching circuit part 31 and the P-side active ground clamp circuit part 23. When the negative voltage is supplied to the ultrasound transducer E, the corresponding control signals are outputted from the transmitting unit 5 in a manner similar to the first embodiment to operate the N-side maximum voltage switching circuit part 33 and the N-side active ground clamp circuit part 24. Consequently, each voltage having a desired waveform can be supplied to the ultrasound transducer E in a manner similar to the first embodiment.

On the other hand, when the positive voltage is supplied to the ultrasound transducer E where the power supply voltage ±HV2 is used, the corresponding control signals are outputted from the transmitting unit 5 in a manner similar to the first embodiment to operate the P-side intermediate voltage switching circuit part 32 and the P-side active ground clamp circuit part 23. When the negative voltage is supplied to the ultrasound transducer E, the corresponding control signals are outputted from the transmitting unit 5 in a manner similar to the first embodiment to operate the N-side intermediate voltage switching circuit part 34 and the N-side active ground clamp circuit part 24. Consequently, each voltage having a desired waveform can be supplied to the ultrasound transducer E in a manner similar to the first embodiment.

### Third Embodiment

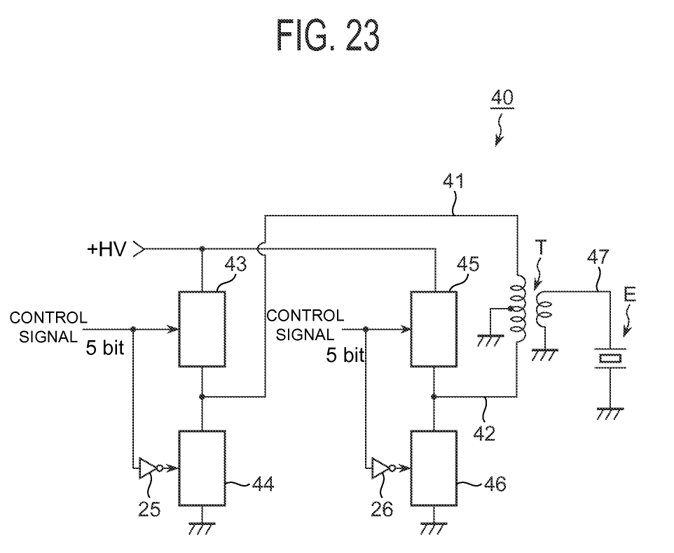

A third embodiment will next be described based on FIG. 23. The same components as those employed in each of the embodiments are, however, respectively given the same reference numerals, and their explanations will therefore be omitted.

A multilevel pulser 40 of an ultrasound transducer drive circuit 10 according to the third embodiment is of a circuit capable of supplying both positive and negative voltages to the ultrasound transducer E only by +HV as the power supply voltage of the power supply unit 12. Described concretely, the multilevel pulser 40 of the present embodiment is provided with a transformer T with an intermediate tap as shown in FIG. 23. A primary-side first output line 41 is connected to one end on the primary side, of the transformer T, and a primary-side second output line 42 is connected to the other end thereof.

The P-side switching circuit part 43 is connected between the power supply voltage +HV and the primary-side first output line 41. The P-side active ground clamp circuit part 44 is connected between the primary-side first output line 41 and ground.

Although not illustrated in particular, the P-side switching circuit part 43 assumes a configuration identical to that of the P-side intermediate voltage switching circuit part 32 employed in the second embodiment and has five switching circuits in which transistors and diodes are connected in series. Although not illustrated in particular, the P-side active ground clam circuit part 44 assumes a configuration identical to that of the P-side active ground clamp circuit part 23 employed in the first embodiment and has five active ground clamp circuits in which diodes and transistors are connected in series.

The P-side switching circuit part 43 is also inputted with each control signal of five bits outputted from the transmitting unit 5. The 5-bit control signals inputted to the P-side switching circuit part 43 are inverted by the corresponding inverter 25, followed by being inputted to the P-side active ground clamp circuit part 44.

The N-side switching circuit part 45 is connected between the power supply voltage +HV and the primary-side second output line 42. Further, the N-side active ground clamp circuit part 46 is connected between the primary-side second output line 42 and ground.

Although not illustrated in particular, the N-side switching circuit part 45 assumes a configuration identical to that of the P-side intermediate voltage switching circuit part 32 employed in the second embodiment and has five circuits in which transistors and diodes are connected in series. Although not illustrated in particular, the N-side active ground clamp circuit part 46 assumes a configuration identical to that of the P-side active ground clamp circuit part 23 employed in the first embodiment.

The N-side switching circuit part 45 is inputted with 5-bit control signals outputted from the transmitting unit 5. The 5-bit control signals inputted to the N-side switching circuit part 45 are inverted by the corresponding inverter 26, followed by being inputted to the N-side active ground clamp circuit part 46.

A secondary-side output line 47 is connected to one end on the secondary side, of the transformer T, and the other end on the secondary side, of the transformer T is connected to ground. The ultrasound transducer E is connected to the secondary-side output line 47. The secondary-side output line 47, the primary-side first output line 41 and the primary-side second output line 42 are one example illustrative of embodiments of output lines employed in the invention.

When the positive voltage is supplied to the ultrasound transducer E in the multilevel pulser 40 of the present embodiment, the corresponding control signals are outputted from the transmitting unit 5 in a manner similar to the first embodiment to operate the P-side switching circuit part 43 and the P-side active ground clamp circuit part 44. When the negative voltage is supplied to the ultrasound transducer E, the corresponding control signals are outputted from the transmitting unit 5 in a manner similar to the first embodiment to operate the N-side switching circuit part 45 and the N-side active ground clamp circuit part 46. Consequently, each voltage having a desired waveform can be supplied to the ultrasound transducer E in a manner similar to the first and second embodiments.

### Fourth Embodiment

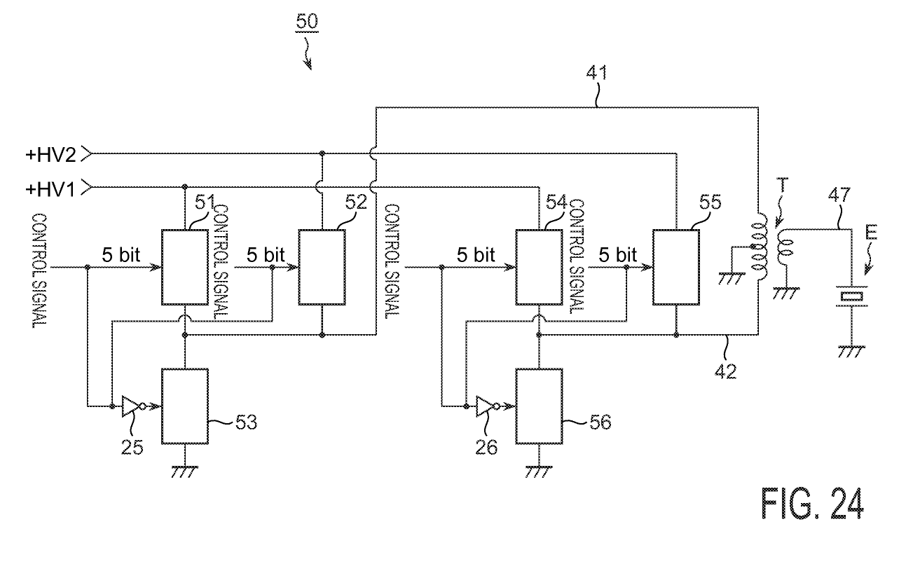

A fourth embodiment will next be described based on FIG. 24. The same components as those employed in each of the embodiments are, however, respectively given the same reference numerals, and their explanations will therefore be omitted.

A multilevel pulser 50 of the ultrasound transducer drive circuit 10 according to the fourth embodiment is of a circuit which is connected to one of different power supply voltages +HV1 and +HV2 in a manner similar to the second embodiment and which is provided with the transformer T and capable of providing both positive and negative voltages to the ultrasound transducer E in a manner similar to the third embodiment.

Described specifically, in the multilevel pulser 50 of the present embodiment, as shown in FIG. 24, a P-side maximum voltage switching circuit part 51 is connected between the power supply voltage +HV1 and the primary-side first output line 41, and a P-side intermediate voltage switching circuit part 52 is connected between the power supply voltage +HV2 and the primary-side first output line 41. Namely, the multilevel pulser 50 of the present embodiment is provided with positive voltage-side switching circuit parts in pairs in a manner similar to the second embodiment. A P-side active ground clamp circuit part 53 is connected between the primary-side first output line 41 and ground.

Although not illustrated in particular, each of the P-side maximum voltage switching circuit part 51 and the P-side intermediate voltage switching circuit part 52 assumes a configuration identical to that of the P-side switching circuit part 43 employed in the third embodiment (i.e., the same configuration as that of the P-side intermediate voltage switching circuit part 32 employed in the second embodiment). Although not illustrated in particular, the P-side active ground clamp circuit part 53 assumes a configuration identical to that of the P-side active ground clamp circuit part 44 employed in the third embodiment (i.e., the same configuration as that of the P-side active ground clamp circuit part 23 employed in the first embodiment).

The P-side maximum voltage switching circuit part 51 and the P-side intermediate voltage switching circuit part 52 are inputted with their corresponding 5-bit control signals outputted from the transmitting unit 5. Each control signal inputted to the P-side maximum voltage switching circuit part 51 or the P-side intermediate voltage switching circuit part 52 is inverted by the corresponding inverter 25, followed by being inputted to the P-side active ground clamp circuit part 53.

An N-side maximum voltage switching circuit part 54 is connected between the power supply voltage +HV1 and the primary-side second output line 42. An N-side intermediate voltage switching circuit part 55 is connected between the power supply voltage +HV2 and the primary-side second output line 42. Namely, the multilevel pulser 50 of the present embodiment is equipped with negative voltage-side switching circuit parts in pairs in a manner similar to the second embodiment. An N-side active ground clamp circuit part 56 is connected between the primary-side second output line 42 and ground.

Although not illustrated in particular, each of the N-side maximum voltage switching circuit part 54 and the N-side intermediate voltage switching circuit part 55 assumes a configuration identical to that of the N-side switching circuit part 45 employed in the third embodiment (i.e., the same configuration as that of the P-side intermediate voltage switching circuit part 32 employed in the second embodiment). Although not illustrated in particular, the N-side active ground clamp circuit part 56 assumes a configuration identical to that of the N-side active ground clamp circuit part 46 employed in the third embodiment (i.e., the same configuration as that of the P-side active ground clamp circuit part 23 employed in the first embodiment).

The N-side maximum voltage switching circuit part 54 and the N-side intermediate voltage switching circuit part 55 are inputted with their corresponding 5-bit control signals outputted from the transmitting unit 5. Each control signal inputted to the N-side maximum voltage switching circuit part 54 or the N-side intermediate voltage switching circuit part 55 is inverted by the corresponding inverter 26, followed by being inputted to the N-side active ground clamp circuit part 56.

When the positive voltage is supplied to the ultrasound transducer E where the power supply voltage +HV1 is used also in the multilevel pulser 50 of the present embodiment, the corresponding control signals are outputted from the transmitting unit 5 in a manner similar to the first embodiment to operate the P-side maximum voltage switching circuit part 51 and the P-side active ground clamp circuit part 53. When the negative voltage is supplied to the ultrasound transducer E, the corresponding control signals are outputted from the transmitting unit 5 in a manner similar to the first embodiment to operate the N-side maximum voltage switching circuit part 54 and the N-side active ground clamp circuit part 56. Consequently, each voltage having a desired waveform can be supplied to the ultrasound transducer E in a manner similar to the first embodiment.

On the other hand, when the positive voltage is supplied to the ultrasound transducer E where the power supply voltage +HV2 is used, the corresponding control signals are outputted from the transmitting unit 5 in a manner similar to the first embodiment to operate the P-side intermediate voltage switching circuit part 52 and the P-side active ground clamp circuit part 53. When the negative voltage is supplied to the ultrasound transducer E, the corresponding control signals are outputted from the transmitting unit 5 in a manner similar to the first embodiment to operate the N-side intermediate voltage switching circuit part 55 and the N-side active ground clamp circuit part 56. Consequently, each voltage having a desired waveform can be supplied to the ultrasound transducer E in a manner similar to the first embodiment.

Although the invention has been explained based on the embodiments as above, it is needless to say that the invention can be changed in various ways within the scope not departing from the gist thereof. For example, the transistors Q1 through Q30 do not need to become the resistance values of 2ⁿ (where n: whole number) times the on resistance taken as a reference, but may be set to 1.8ⁿ to 2.2ⁿ (where n: whole number) times the on resistance taken as the basic.

The respective modifications described in the first embodiment can similarly be applied to the second through fourth embodiments.

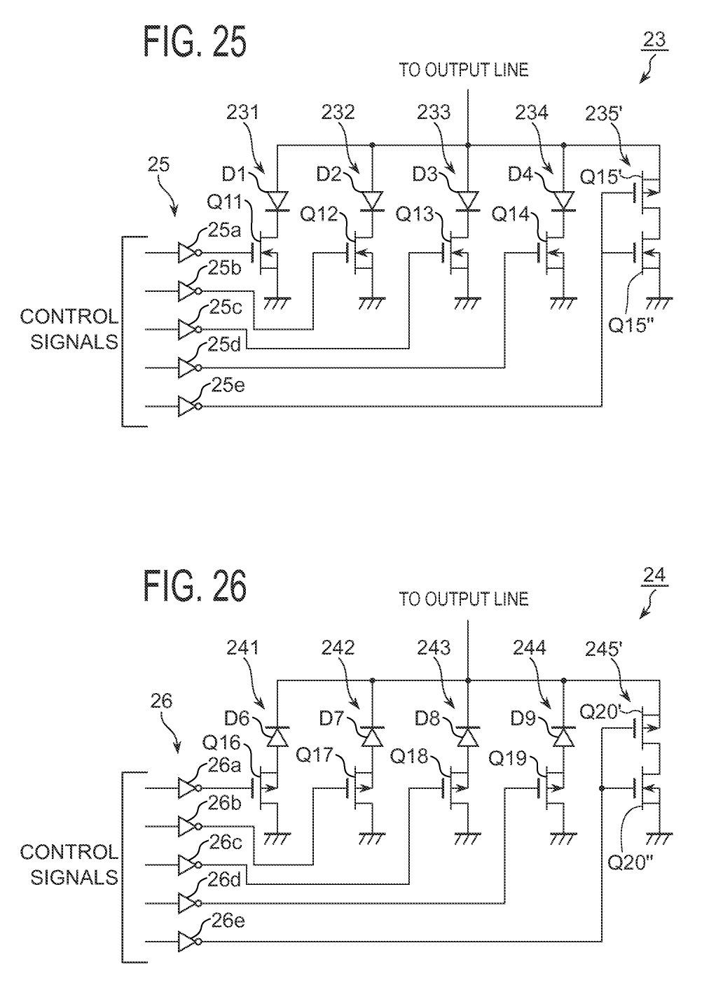

When the P-side active ground clamp circuit part 23 shown in FIG. 6 and the N-side active ground clamp circuit part 24 shown in FIG. 7 are used, the output voltage of the output line 20 is brought to a voltage obtained by superimposing residual potentials due to the diodes D5 and D10 on a ground potential. Thus, in order to provide a ground potential that does not contain such residual potentials, any of the P-side active ground clamp circuits 231 through 235 (inclusive of the case where they configure the P-side active ground clamp circuit parts 44 and 53 and the N-side active ground clamp circuit parts 46 and 56 in the third and fourth embodiments), and any of the N-side active ground clamp circuits 241 through 245 may be configured by p channel and n channel FETs connected in series. As shown in FIG. 25, for example, a fifth P-side active ground clamp circuit 235′ may be configured by a circuit in which a transistor Q15′ comprised of a p channel FET and a transistor Q15″ comprised of an n channel FET. Further, as shown in FIG. 26, a fifth N-side active ground clamp circuit 245′ may be configured by a circuit in which a transistor Q20′ comprised of a p channel FET and a transistor Q20″ comprised of an n channel FET are connected in series.

When the P-side active ground clamp circuit part 23 is operated, the transistors Q15′ and Q15″ are turned on and off in sync with each other. Further, when the N-side active ground clamp circuit part 24 is operated, the transistors Q20′ and Q20″ are turned on and off in sync with each other. Consequently, the output voltage of the output line 20 can be brought to the ground potential that does not contain the residual potentials.

In each of the embodiments, when the output voltage of the output line 20 is set to the ground potential, any of the circuits that configure the P-side active ground clamp circuit parts 23, 44 and 55, and any of the circuits that configure the N-side active ground clamp circuit parts 24, 46 and 56 may be turned on together. It is thus possible to suppress voltage overshoot when the output voltage of the output line 20 reaches the ground potential.

Many widely different embodiments of the invention may be configured without departing from the spirit and the scope of the present invention. It should be understood that the present invention is not limited to the specific embodiments described in the specification, except as defined in the appended claims.

1. An ultrasound transducer drive circuit comprising:
an output line connected to an ultrasound transducer;
a plurality of switching circuits each configured to selectively enable a connection of a power supply unit with the output line;
a plurality of ground clamp circuits each configured to selectively enable a connection of the output line with ground; and
a control signal output unit configured to output control signals to selectively enable the plurality of switching circuits and the plurality of ground clamp circuits, wherein the plurality of switching circuits are different in impedance from one another, and the plurality of ground clamp circuits are different in impedance from one another.

2. The ultrasound transducer drive circuit according to claim 1,
wherein the plurality of switching circuits are connected in parallel to one another,
wherein the plurality of ground clamp circuits are connected in parallel to one another and also connected in parallel to an ultrasound transducer, and
wherein the plurality of switching circuits and the plurality of ground clamp circuits are respectively connected in series with one another.

3. The ultrasound transducer drive circuit according to claim 1, wherein the respective impedances of the plurality of switching circuits and the respective impedances of the plurality of ground clamp circuits are set such that a voltage of the output line has linearity with respect to each of the control signals.

4. The ultrasound transducer drive circuit according to claim 2, wherein the respective impedances of the plurality of switching circuits and the respective impedances of the plurality of ground clamp circuits are set such that a voltage of the output line has linearity with respect to each of the control signals.

5. The ultrasound transducer drive circuit according to claim 1, wherein the impedances of the plurality of switching circuits and the impedances of the plurality of ground clamp circuits are respectively set to 1.8ⁿ to 2.2ⁿ (where n: whole number) times the impedance of a first switching circuit taken as a reference and the impedance of a first ground clamp circuit taken as a reference.

6. The ultrasound transducer drive circuit according to claim 2, wherein the impedances of the plurality of switching circuits and the impedances of the plurality of ground clamp circuits are respectively set to 1.8ⁿ to 2.2ⁿ (where n: whole number) times the impedance of a first switching circuit taken as a reference and the impedance of a first ground clamp circuit taken as a reference.

7. The ultrasound transducer drive circuit according to claim 1, wherein the impedance of any of the plurality of switching circuits and the impedance of any of the plurality of ground clam circuits are equal to each other.

8. The ultrasound transducer drive circuit according to claim 2, wherein the impedance of any of the plurality of switching circuits and the impedance of any of the plurality of ground clam circuits are equal to each other.

9. The ultrasound transducer drive circuit according to claim 1, wherein the control signal output unit outputs is configured to output the control signals such that a first switching circuit of the plurality of switching circuits and a first ground clamp circuit of the plurality of ground clamp circuits each having equal orders of magnitude of impedance are enabled, and the remaining switching circuits and the remaining ground clamp circuits are disabled.

10. The ultrasound transducer drive circuit according to claim 2, wherein the control signal output unit is configured to output the control signals such that a first switching circuit of the plurality of switching circuits and a first ground clamp circuit of the plurality of ground clamp circuits each having equal orders of magnitude of impedance are enabled, and the remaining switching circuits and the remaining ground clamp circuits are disabled.

11. The ultrasound transducer drive circuit according to claim 1, further comprising a correction device configured to set a voltage of the output line to a desired voltage.

12. The ultrasound transducer drive circuit according to claim 11, wherein the correction device comprises one of a lookup table and a correction computer configured to set each control signal outputted from the control signal output unit as a control signal capable of obtaining a desired output voltage and to output the respective control signal to each of the plurality of switching circuits and the plurality of ground clamp circuits.

13. The ultrasound transducer drive circuit according to claim 1, further comprising:
a plurality of transistors each used as a switching element of each of the plurality of switching circuits; and
a correction device comprising a plurality of sub-transistors connected in parallel with a corresponding transistor as a subarray and smaller in on resistance than the corresponding transistor.

14. The ultrasound transducer drive circuit according to claim 1, wherein at least one of the plurality of ground clamp circuits is always brought to disabled state.

15. The ultrasound transducer drive circuit according to claim 1, further comprising a plurality of sets of switching circuit parts each comprising a plurality of the switching circuits different in impedance, wherein the plurality of switching circuit parts are respectively inputted with different power supply voltages each corresponding to an ultrasound transmission mode.

16. An ultrasound diagnostic apparatus comprising:
an ultrasound probe comprising at least one ultrasound transducer; and
ultrasound transducer drive circuit comprising:
an output line connected to the ultrasound transducer;
a plurality of switching circuits each configured to selectively enable a connection of a power supply unit with the output line;
a plurality of ground clamp circuits each configured to selectively enable a connection of the output line with ground; and
a control signal output unit configured to output control signals to selectively enable the plurality of switching circuits and the plurality of ground clamp circuits, wherein the plurality of switching circuits are different in impedance from one another, and the plurality of ground clamp circuits are different in impedance from one another.

17. The ultrasound diagnostic apparatus according to claim 16,
wherein the plurality of switching circuits are connected in parallel to one another,
wherein the plurality of ground clamp circuits are connected in parallel to one another and also connected in parallel to an ultrasound transducer, and
wherein the plurality of switching circuits and the plurality of ground clamp circuits are respectively connected in series with one another.

18. The ultrasound diagnostic apparatus according to claim 16, wherein the respective impedances of the plurality of switching circuits and the respective impedances of the plurality of ground clamp circuits are set such that a voltage of the output line has linearity with respect to each of the control signals.

19. The ultrasound diagnostic apparatus according to claim 16, wherein the impedances of the plurality of switching circuits and the impedances of the plurality of ground clamp circuits are respectively set to 1.8ⁿ to 2.2ⁿ (where n: whole number) times the impedance of a first switching circuit taken as a reference and the impedance of a first ground clamp circuit taken as a reference.

20. The ultrasound diagnostic apparatus according to claim 16, wherein the impedance of any of the plurality of switching circuits and the impedance of any of the plurality of ground clamp circuits are equal to each other.
